(19) United States
(12) Patent Application Publication
(10) Pub. No.: US 2014/0267358 A1
(43) Pub. Date: Sep. 18, 2014

(54) Method and Apparatus for Displaying Periodic Signals and Quasi-Periodic Signals Generated by a Medical Device

(71) Applicant: GestlnTime Inc., Palo Alto, CA (US)

(72) Inventors: Rohit MITTAL, Sunnyvale, CA (US); Krishna Garimella, San Jose, CA (US); Suresh Subramaniam, Palo Alto, CA (US)

(21) Appl. No.: 13/913,313

(22) Filed: Jun. 7, 2013

Related U.S. Application Data
(63) Continuation-in-part of application No. 13/838,563, filed on Mar. 15, 2013.

(51) Int. Cl. A61B 5/00; G06T 11/20

(52) U.S. Cl. CPC A61B 5/742; A61B 5/7235; G06T 11/206
USPC 345/589; 345/440

(57) ABSTRACT

A method and apparatus for displaying periodic signals generated by a medical device is disclosed. A method and apparatus for displaying quasi-periodic signals generated by a medical device also is disclosed.

### PRIORITY CLAIM

This is a continuation-in-part of U.S. application Ser. No. 13/838,563, titled “Method and Apparatus for Displaying Periodic Signals Generated by a Medical Device” and filed on Mar. 15, 2013, which is incorporated herein by reference in its entirety.

### TECHNICAL FIELD

A method and apparatus for displaying periodic signals generated by a medical device is disclosed. A method and apparatus for displaying quasi-periodic signals generated by a medical device also is disclosed.

### BACKGROUND OF THE INVENTION

Electrocardiogram (EKG, also known as ECG) devices are well-known in the prior art. They measure the electrical activity of the human heart using electrodes and create tracings of the activity on paper or on a visual display.

FIG. 1 depicts a prior art medical device 10 along with output 20. In this particular exemplary depiction, medical device 10 is an EKG device and output 20 is EKG data. Notably, output 20 comprises either a graph printed on a scroll of paper or a graphical display on a screen that scrolls in real-time as the electrical activity is measured. Using prior art device 20, a doctor or medical professional must read the scroll of paper or watch the tracings on a screen in real time. This can be a tedious and challenging exercise that contains the inherent risk that the doctor or medical professional will miss an important change in the monitored activity.

Many medical devices create periodic signals as well that represent activity within the human body. For example, medical devices exist in the areas of electromyography (EMG) (to monitor muscle activity), electroencephalography (EEG) (to monitor brain activity), polysomnography (to monitor breathing activity during sleep), and other areas in which periodic signals are generated and monitored in real-time by a doctor or medical professional.

In the electrical engineering field, oscilloscopes and other tools are well-known for displaying electrical signals on a screen. One technique used by such tools is to create an “eye diagram” for periodic signals. The technique involves superimposing the signal from one period over the signal from the next period and the next period, and so on. An exemplary eye diagram 30 is shown in FIG. 2. This allows the user to physically see multiple periods of the signal at one time in a limited amount of space and to readily view any differences or deviations in the signals.

What is needed is a device for generating an eye diagram for periodic signals generated by medical devices and to identify any excursions from the mean values, expected values, or other thresholds. What is further needed is the ability to examine an excursion in more detail and to quickly see the data before and after the excursion occurred.

What is further needed is the ability to apply these concepts to quasi-periodic signals generated by medical devices.

### SUMMARY OF THE INVENTION

The aforementioned problem and needs are addressed through an embodiment for generating an eye diagram of a periodic signal output from a medical device and for examining an excursion in more detail. Another embodiment provides the same benefit for quasi-periodic signals.

### BRIEF DESCRIPTION OF THE DRAWINGS

FIG. 1 depicts a prior art medical device and its output.

FIG. 2 depicts a prior art eye diagram.

FIG. 3 depicts an embodiment for generating an eye diagram using a periodic signal from a medical device.

FIG. 4 depicts an embodiment for identifying and capturing one or more periods of data from a periodic signal from a medical device.

FIG. 5 depicts an embodiment for generating an eye diagram using a periodic signal where the eye diagram shows an excursion in the signal.

FIG. 6 depicts an embodiment for displaying an expanded version of the periodic signal in response to a user instruction after viewing the eye diagram of FIG. 5.

FIG. 7 depicts an embodiment for generating an eye diagram using a quasi-periodic signal from a medical device.

FIG. 8 depicts an embodiment for generating an eye diagram using a quasi-periodic signal where the eye diagram shows an excursion in the signal.

FIG. 9 depicts an embodiment for displaying an expanded version of the quasi-periodic signal in response to a user instruction after viewing the eye diagram of FIG. 8.

FIG. 10 depicts various display options for the eye diagram.

FIG. 11 depicts an embodiment of display eyewear.

### DETAILED DESCRIPTION OF THE PREFERRED EMBODIMENTS

An embodiment will now be described with reference to FIG. 3. Medical device 10 is the same prior art device described previously with reference to FIG. 1. The output of medical device 10 is provided as an input to processing device 40. In this particular example, the output is EKG data, but the same principles apply to any periodic data collected from a medical device.

In one embodiment, processing device 40 is a computing device (such as a desktop, notebook, server, tablet, mobile device, or other computer) comprising a processor, memory, non-volatile storage (such as a hard disk drive or flash memory array), I/O connection (such as a USB connection) for communicating with a medical device, and an I/O connection for sending output to a display, printer, or other device. Optionally, processing device 40 can itself include a display (as might be the case if processing device 40 is a tablet or mobile device). Processing device 40 comprises software code for performing the functions described herein.

Processing device 40 receives the periodic signal generated by medical device 10 and generates an output 50 that comprises an eye diagram of the signal by superimposing one period of the signal on top of another period of the signal, and so on. One of ordinary skill in the art will appreciate that output 50 is much easier to read and analyze than output 20 shown in FIG. 1.

The periodic signal generated by medical device 10 can be either analog or digital. If the periodic signal is an analog signal, processing device 40 will perform analog-to-digital conversion using known techniques. If the periodic signal already is a digital signal, then no conversion is needed.

FIG. 4 depicts a method for generating an eye diagram as shown in FIG. 3. Processing device 40 stores digital data in a buffer (step 200), where the buffer is contained within memory. The digital data is the data received from medical device 10 (if the data is digital) or is the digitized version of the data received from medical device 10 (if the data is analog). In the example of EKG data, the digital data represents the electrical impulses coming out of the heart Processing device 40 identifies peak values within a sequence of digital data stored in the buffer (step 210). This can be done simply by comparing all data points received within a time period t₁ that is several times larger than the expected period of a normal heartbeat. For example, t₁ can be 5 seconds. The peak value of each heartbeat will be approximately the same. Processing device 40 determines the number of data points B between peak values to determine the period of the data (step 220). The value of B will depend upon the patient's heart and the sampling rate of medical device 10 (if it generates digital data) or the sampling rate of the analog-to-digital converter of processing device 40 (if medical device 10 generates analog data). Processing device 40 optionally resamples the data to collect N data points per period (step 230). This might be desirable, for instance, if B is not a power of 2 (which is likely). For example, if B is 1157 (representing 1157 data points per period), one could choose N to be 1024, where 1024 data points are collected by resampling the B data points using known digital sampling techniques. Processing device 40 displays R periods of data on output 50 (step 240), where R is any integer value that represents the number of periods of data displayed in the eye-diagram at any given time. R optionally can be a very large number such that all of the data will be displayed on the eye-diagram.

A baseline sequence representing one heart beat can be utilized. The baseline sequence can represent an ideal heart beat that is stored in non-volatile storage of processing device 40, or the baseline sequence can be determined based on data collected from the patient's heart beat. For example, once processing device 40 has stored multiple periods of data for the patient's heart beat, it can determine the mean value for each data location within the sequence of data in one period over X periods of data. If X=50 and N=1024, for instance, processing device 40 will determine the mean value at each data location aᵢ (where i ranges from 1 to N or 1024 in this example) within 50 periods of data. The resulting sequence a will represent the baseline heartbeat.

Once a baseline is determine, excursions can be automatically identified in the data obtained from medical device 10. If we assume N is 1024, then each period will have 1024 data points, and the baseline sequence aᵢ will also have 1024 data points. A threshold L can be set, where L is a percentage of deviation. Each data point dₕᵢ (where h ranges from 1 to T and T represents the number of periods of data captured to date, and i ranges from 1 to N, and i represents the location within the sequence as is the case with aᵢ) is compared to aᵢ. If dₕᵢ is 1% greater or less than aᵢ, then dₕᵢ represents an excursion.

All data points representing excursions are recorded or flagged by processing device 40. For example, processing device 40 can maintain a data structure for each data point dₕᵢ that includes a flag bit, where a 0 represents no excursion and a 1 represents an excursion. In the alternative, processing device 40 can maintain a list of each data point dₕᵢ that is an excursion.

An embodiment is now shown in FIG. 5. FIG. 5 is similar to FIG. 3 except output 50 shows an graphical excursion 60 in one period of the signal. Graphical excursion 60 represents a deviation from the “norm” as shown in the eye diagram and comprises data points dₕᵢ that were determined to be excursions, for example, by using the method described above. One of ordinary skill in the art will understand that graphical excursion 60 is much easier to identify than it would have been in the traditional tracings on a scroll of paper or tracings displayed on a screen that scrolls in real-time.

Optionally, when an excursion is identified, processing device 40 can generate alert 70. Alert 70 can appear on the display as part of output 50, or it can be sent over email, SMS or MMS message, a phone call, a web-based message, etc. Processing device 40 can generate alert 70 based on any of the following: identification of an excursion as described above; statistically significant deviation from the mean value of the periodic signal at that location within the period; significant deviation from the expected value of the signal for a healthy individual; or a value above a pre-determined threshold specified by the user or programmed into processing device 40.

Optionally, processing device 40 can enable a user to request more information regarding graphical excursion 60 or any other portion of the eye diagram contained in output 50. Such requests can be made through a mouse click on a display, through a keyboard, or using a voice command.

If a user requests further information regarding graphical excursion 60 (such as by clicking on it using a mouse and a display), then optionally a traditional view will be created as shown in FIG. 6.

In FIG. 6, processing device 40 generates output 70, which resembles a traditional display of periodic signal. Graphical excursion 60 is shown, and the selected period 80 in which graphical excursion 60 appears is highlighted for the user, such as by drawing a box around the relevant portion of the signal as shown in FIG. 6, altering the color or brightness of that portion of the signal, or otherwise changing the appearance of that portion of the signal. The amount of data to be displayed before and after the excursion can be user controlled. Less amount of data display can lead to faster viewing whereas larger amount of data can be slower and appear cluttered on a limited viewing screen.

One of ordinary skill in the art will understand that this combination of the prior art medical devise with the prior art eye diagram technique yields an invention that will enhance the ability of doctors and other medical professionals to analyze periodic signal from medical devices, such as EKG or ECG data, and to quickly identify any troublesome excursions in the data.

The embodiments described thus far have utilized periodic signals generated by medical device 10. Many of the same principles can be applied to quasi-periodical signals generated by medical device 15. A quasi-periodic signal is a signal that represents measurements that are not periodic by nature (such as blood pressure, weight, blood sugar, etc.) but which are captured on a periodic basis (such as a measurement taken daily at 8 am or every few hours in a day).

An embodiment is shown in FIG. 7. In FIG. 7, medical device 15 captures data from a patient that is not periodic in nature. Examples of medical device 15 include a scale to measure weight, a sphygmomanometer to measure blood pressure, a glucometer to measure blood sugar levels.

Medical device 15 transmits data to processing device 40, which is the same processing device 40 described with reference to other embodiments. Processing device 40 records the data, which in this example, comprises date/time and value information. For example, if medical device 15 is a scale, the data might be: 5-28-13 at 0801, 155 pounds. Over time, processing device 40 organizes the data into quasi-periodic groups. For instance, if processing device 40 receives a certain type of reading at approximately the same time each day, it will organize the data into a data structure and can optionally generate output 55 that depicts the readings of, for example, a patient's weight at 8 am on a daily basis. Even if the data is not obtained on a completely regular basis, for example at 8 am on one day, 10 am on another day etc., the dataset will still be assumed to be quasi-periodic. When the number of readings becomes too large to display on a single screen, the data can be shown as an eye-diagram as shown in FIG. 7.

As with previous embodiments, a baseline can be generated (for example, by averaging the first F values), and processing device 40 can identify excursions from the baseline. The same methodology described previously can be used. This is depicted in FIG. 8, where graphical excursion 60 is shown in output 75.

With reference to FIG. 9, as with previous embodiments, a user can request further information about graphical excursion 60, and once this occurs, the graphical excursion 60 and data that preceded and followed the graphical excursion will be displayed as output 75, and the graphical excursion 60 can be highlighted for the user.

FIG. 10 depicts various mechanisms for a user to view output 50, output 55, output 70, output 75, and other output that can be utilized for the embodiments described previously with reference to FIGS. 3-9. These mechanisms include a display 100 (such as an LCD), mobile device 110 (such as a tablet or mobile phone), and eyewear 120.

FIG. 10 depicts an example of eyewear 120. Eyewear 120 comprises lenses 122 and frame 121 (just as with normal glasses). But it also includes display unit 130 and processing and transmission unit 140 (embedded within the frame 121).

An example of eyewear 120 was recently announced by Google as the “Google Glass” product. Eyewear 120, such as the Google Glass, comprises a display unit 130 that displays data that you could otherwise display on an LCD or other display. Display unit 130 can be used to display the eye diagrams discussed previously.

The possible uses of eyewear 120 by physicians in conjunction with the display of periodic signals discussed above are numerous. For example, a physician could: (a) view a periodic signal during a patient examination, during a remote consultation, or during a collaborative session with a fellow physician (e.g., two physicians viewing the same EKG); (b) look at the patient in the physician's office while the display unit 130 displays a periodical signal; or (c) apply physical pressure to the patient or perform other techniques or tests and get instant visual feedback regarding the effect on heartbeat, etc.

References to the present invention herein are not intended to limit the scope of any claim or claim term, but instead merely make reference to one or more features that may be covered by one or more of the claims. Materials, processes and numerical examples described above are exemplary only, and should not be deemed to limit the claims. It should be noted that, as used herein, the terms “over” and “on” both inclusively include “directly on” (no intermediate materials, elements or space disposed there between) and “indirectly on” (intermediate materials, elements or space disposed there between). Likewise, the term “adjacent” includes “directly adjacent” (no intermediate materials, elements or space disposed there between) and “indirectly adjacent” (intermediate materials, elements or space disposed there between). For example, forming an element “over a substrate” can include forming the element directly on the substrate with no intermediate materials/elements there between, as well as forming the element indirectly on the substrate with one or more intermediate materials/elements there between.

What is claimed is:

1. A system for processing and displaying a quasi-periodic signal from a medical device, comprising:
a medical device for generating a quasi-periodic signal;
a processing device for receiving the quasi-periodic signal and generating an eye diagram based on the quasi-periodic signal;
a display coupled to the processing device for displaying the eye diagram.

2. The system of claim 1, wherein the medical device is an scale.

3. The system of claim 1, wherein the medical device is a sphygmomanometer.

4. The system of claim 1, wherein the medical device is a glucometer.

5. The system of claim 1, wherein the processing device is configured to generate an alert if the received data at a location within a period of the quasi-periodic signal exceeds the mean of prior data at that location.

6. The system of claim 1, wherein the processing device is configured to generate an alert if the received data at a location within a period of the quasi-periodic signal exceeds a pre-determined threshold.

7. The system of claim 1, wherein the processing device is capable of receiving a request from a user relating to the quasi-periodic signal.

8. The system of claim 7, wherein the processing device is capable of highlighting a portion of the quasi-periodic signal in response to the request.

9. The system of claim 1, wherein the display is contained within eyewear.

10. A method for displaying a quasi-periodic signal from a medical device, comprising:
receiving a quasi-periodic signal from a medical device;
generating an eye diagram based on the quasi-periodic signal; and
displaying the eye diagram on a display.

11. The method of claim 10, wherein the medical device is a scale.

12. The method of claim 10, wherein the medical device is a sphygmomanometer.

13. The method of claim 10, wherein the medical device is a glucometer.

14. The method of claim 10, further comprising generating an alert if the received data at a location within a period of the quasi-periodic signal exceeds the mean of prior data at that location.

15. The method of claim 10, further comprising generating an alert if the received data at a location within a period of the quasi-periodic signal exceeds a pre-determined threshold.

16. The method of claim 10, further comprising receiving a request from a user relating to the quasi-periodic signal.

17. The method of claim 16, wherein the processing device is capable of highlighting a portion of the quasi-periodic signal in response to the request.

18. The method of claim 10, wherein the display is contained within eyewear.

19. A method for processing and displaying data from a medical device, comprising:
storing data comprising a plurality of data points received from a medical device;
identifying, by a processing device, a plurality of peak values in the data;
determining, by the processing device, the period of the data based on the number of data points between the peak values; and
displaying R periods of data on a display, where R is an integer greater than one.

20. The method of claim 19, further comprising:
resampling the data to collect N data points per period, where N is an integer greater than one.
